(19) United States
(12) Patent Application Publication
(10) Pub. No.: US 2002/0058613 A1
(43) Pub. Date: May 16, 2002

(54) Methods of controlling axonal growth

(75) Inventors: Gerald E. Schneider, Brookline, MA (US); Dong Feng Chen, Brookline, MA (US); Susumu Tonegowa, Chestnut Hill, MA (US); Sonal Jhaveri, Brookline, MA (US)

Correspondence Address: LAHIVE & COCKFIELD, 28 STATE STREET, BOSTON, MA, 02109, US

(73) Assignee: Massachusetts Institute of Technology

(21) Appl. No.: 10/035,376

(22) Filed: Nov. 1, 2001

Related U.S. Application Data
(63) Continuation of application No. 08/816,371, filed on Mar. 13, 1997, now abandoned.
Application No. 08/816,371 is a continuation-in-part of application No. 08/713,423, filed on Sep. 13, 1996, now abandoned.
(60) Provisional application No. 60/021,713, filed on Jul. 12, 1996.

(51) Int. Cl. A61K 38/00; A01N 37/18

(52) U.S. Cl. 514/2; 514/12

(57) ABSTRACT

Agents which modulate a bcl family member to control axonal growth and regeneration are described. These bcl modulating agents promote axonal growth and regeneration in the neural cells of a subject. Compositions for promoting axonal cell growth in a subject also are described. The compositions of the present invention include an effective amount of an agent which modulates a bcl family member and in a pharmaceutically acceptable carrier. Other described aspects include packaged drugs for treating a state characterized by diminished potential for axonal growth. The packaged compounds and agents also include instructions for using the agent to promote axonal growth in a subject.

### RELATED APPLICATIONS

The present application is a continuation-in-part application of U.S. Ser. No. 08/713,423 filed on Sep. 13,1996, entitled METHODS OF CONTROLLING AXONAL GROWTH. Both this application and the aforementioned application claim priority to a provisional application, U.S. Ser. No. 60/021,713 filed on Jul. 12, 1996. The contents of both applications are expressly incorporated by reference.

### GOVERNMENT FUNDING

Work described herein was supported, in part, by a grant awarded by the National Institutes of Health. The U.S. government may therefore have certain rights in this invention.

### BACKGROUND OF THE INVENTION

The functions of the brain and spinal cord depend on cells called neurons, which contact and communicate with each other through nerve fibers called axons. Injuries to the brain or spinal cord can cause the loss of many axons and the disruption of connections between neurons in the brain and spinal cord. This disruption results in the devastating loss of function in patients with such injuries, leaving them with varying degrees of paralysis and losses in sensory or cognitive functions. Some of these losses are permanent since there is very little regeneration of these axons in mammals.

Most neurons of the mammalian central nervous system (CNS) lose the ability to regenerate severed axons after a certain point in development (Aubert, I., et al. Curr. Opin. Biol. 5, 625-635 (1995); Bähr, M. & Bonhoeffer, F. TINS 17, 473-479 (1994). Acutely damaged CNS neurons do, however, make an abortive attempt at regenerating. It has been suggested that axotomized neurons in the CNS are able to produce new axons, as in the peripheral nervous system (PNS), but that regeneration fails because of the non-permissive nature of the environment in which the new growth cones are formed (Breckness and Fawcett. Biol. Rev. 71:227 (1996)). Early work suggested that the nonpermissive CNS environment resulted from the lack of chemical factors which were present in the PNS (Cajal. Degeneration and Regeneration of the Nervous System, Oxford University Press, Oxford (1928)). Among the molecules thought to be important in axonal regeneration are the neurotrophins, which include: nerve growth factor (NGF), brain-derived neurotrophic factor (BDNF), neurotrophin-3 (NT-3), NT-4/5, and NT-6 (Silos-Santiago et al. Curr. Opin. Neurobiol. 5:42 (1995); Davies. TINS 18:355(1995)). The receptors of the Trk family are thought to play key roles in the mechanism of action of neurotrophins (Greene and Kaplan. Curr. Opinion in Neurobiol. 5:579 (1995)). Other non-neurotrophin growth factors are thought to influence neuronal populations, including: ciliary neurotrophic factor (CNTF), leukemia inhibitory factor (LIF), insulin-like growth factor (IGF)-I and IGF-II, glial cell line-derived neurotrophic factor (GDNF), growth promoting activity (GPA), basic fibroblast growth factor (bFGF) and members of the transforming growth factor b (TGFβ) superfamily (Silos-Santiago et al.; Davies supra). Apolipoprotein E, and laminin are also thought to play a role in axonal regeneration (Breckness and Fawcett, supra). The mature CNS, however, is not devoid of all of these factors. Another explanation for the failure of axonal regeneration in the CNS has been that the CNS contains inhibitors of axonal growth, such as proteins found in the membranes of oligodendrocytes and CNS myelin (Schnell, L. & Schwab, M. E. Nature 343, 269-272 (1990)).

More recent evidence, however, indicates that the ability of embryonic neurons to develop axons may be a property of the neurons themselves. For example, embryonic neurons are better at growing axons than adult neurons are at regenerating them, even when those embryonic neurons are placed in an adult CNS environment. Embryonic neurons transplanted into the adult CNS are able to form long axons, even along myelinated tracts (Wictorin et al., Nature 347:556 (1990); Davies et al. Journal of Neurosciences 14:1596(1994)).

One protein which has been implicated in axonal growth is GAP-43. A correlation has been found between the expression of GAP-43 (also known as B50, pp46, neuromodulin, and F1) and the ability of a neural cell to regenerate an axon. GAP-43 is a phosphoprotein found in neuronal growth cones, which has been found to bind to calmodulin (Spencer and Willard. Exp. Neurol. 115:167 (1991)) and to stimulate nucleoside triphosphate binding to the G protein, G_{O} (Strittmatter et al. Nature 344:836 (1990)). While the relationship between the synthesis of GAP-43 and periods of axon extension, has suggested its role in axonal growth (Fidel et al. Soc. Neurosci. Abstr. 16:339(1990); Schotman et al., Soc. Neruosci. Abstr. 16:339(1990)), some axotomized RGCs have been shown to up-regulate GAP-43 without regenerating (Doster et al. Neuron 6:635(1991; Schaden et al., Journal of Neurobiology 25:1570(1994)). Moreover, PC12 cells have been shown to extend neurites in the absence of GAP-43 (Baetge and Hammang. Neuron 6:21(1991)).

The bcl-2 gene was discovered at the breakpoint region of the t(14; 18) chromosomal translocation. Bcl-2 is a 26 kD integral membrane protein that has been localized to the outer mitochondrial membrane, perinuclear membrane and smooth endoplasmic reticulum, and has been shown to be important in the regulation of apoptosis (Nunez et al. Immunology Today 15:583(1994)). Apoptosis is also known as “programmed cell death” and involves the activation in cells of a genetic program leading to cell death. Apoptosis occurs in both normal cell development and certain disease states. For example, downregulation of bcl-2 is a common feature of normal lymphoid populations undergoing programmed cell death and selection, whereas upregulation of bcl-2 appears to be part of the positive selection mechanism (Nunez et al. supra). The death of neurons which occurs in Alzheimer's dementia and Parkinson's disease, as well as in cancer and viral infection, also shows the hallmarks of apoptosis. Thus, the use of bcl-2 to treat neurodegenerative diseases of the CNS which are characterized by apoptosis has been proposed (WO 94/27426).

### SUMMARY OF THE INVENTION

The present invention is based, at least in part, on the discovery that bcl-2 plays a role in the growth and/or regeneration of axons in neural cells. The present invention pertains to a method of promoting axonal growth in a neural cell. The method involves modulating the expression or bioactivity of a bcl family member in a neural cell such that axonal growth occurs.

The invention further pertains to methods of treating a subject for a state characterized by diminished potential for axonal growth. The method involves administering a therapeutically effective amount of an agent which modulates the bioactivity or expression of a bcl family member in a subject such that axonal growth occurs. In one embodiment, the agent is a a gene construct for expressing a bcl family member. The gene construct is formulated for delivery into neural cells of the subject such that axonal growth occurs.

Other aspects of the invention include pharmaceutical preparations and packaged drugs used in the aforementioned methods. Methods for selecting agents or bcl family members for use within the aforementioned methods also are part of this invention.

### BRIEF DESCRIPTION OF THE FIGURES

FIG. 1. The expression of bcl-2 is essential for the growth of most retinal axons in culture: Retinal axon growth was quantitated in cultures from wild-type (C57BL/6J), bcl-2 null mice, and bcl-2 transgenic mice. (A) Quantification of cultures derived from embryonic day 15 pups genetically deficient in bcl-2: retinal explant derived from heterozygous (+/−) or homozygous (−/−) mutant mice both showed decreased numbers of axons that invaded the tectal tissue when-compared with those of wild-type animals (+/+) at this age. (B) Growth of retinal axons from adult retinae was quantitated. Retinal explants derived from adult transgenic mice display 10-fold more axonal growth into E16 tectum than into comparable tissues from wild-type mice. (C) Growth curves of retinal axons obtained from retinotectal cocultures, using tissues from wild-type or transgenic animals aged embryonic day 14 through day 5 after birth. Mouse genotype was determined by genomic Southern or PCR analysis of genomic DNA isolated from the mouse tails. Data obtained from wild-type mice are plotted with the solid line, and those from transgenic mice are depicted by the dotted line. Note that at age E18 or older, there is a marked decrease in numbers of retinal axons from wild-type animals. This decline was not observed for bcl-2 transgenic mice.

FIG. 2. ZVAD (Z-Val-Ala-Asp-CH₂F, Enzyme Systems Products), though sufficient to prevent death of RGCs, is not sufficient to promote axonal growth: This figure shows the effects of the ICE-like protease inhibitor, ZVAD, on the survival and neurite outgrowth of RGCs in culture. (A) Shows the numbers of surviving RGCs in dissociated retinal cell cultures treated with different doses of ZVAD. Doses from 0 to 200 M were tested. (B) Shows the quantification of cell death in retinal explants from ZVAD-treated retinotectal cocultures. Three doses of ZVAD (50, 100, and 200M) were examined, and cultures were prepared from 2 day old wild-type animals. (C) Quantification of retinal axon growth in coculture experiments parallel to those in (B). Note that by increasing the concentration of ZVAD, the number of dying cells in retinal explants decreased, whereas, the number of growing axons did not change significantly.

### DETAILED DESCRIPTION OF THE INVENTION

The present invention provides for methods of promoting axonal growth in a neural cell. The methods involve modulating the expression or bioactivity of a bcl family member.

As used herein, the term “axonal growth” refers to the ability of a bcl modulating agent to enhance the extension (e.g., regeneration) of axons and/or the reestablishment of nerve cell connectivity. Axonal growth as used herein is not intended to include within its scope all neurite sprouting nor is it intended to include the promotion of neural cell survival through means other than the promotion of axonal growth. For example, axonal growth is intended to include neurite sprouting which occurs after an axon is damaged and neurite sprouting which occurs in conjunction with the extension of the axon. Axonal growth as used herein includes axonal regeneration in severed neurons which occurs at, or near, the site at which the axon was severed.

The term “neural cell” as used herein is meant to include cells from both the central nervous system (CNS) and the peripheral nervous system (PNS). Exemplary neural cells of the CNS are found in the gray matter of the spinal cord or the brain and exemplary neural cells of the PNS are found in the dorsal root ganglia.

The term “bcl family member” or “bcl polypeptide” as used in the instant application is meant to include polypeptides, such as bcl-2 and other members of the bcl family. Bcl family member is meant to include within its scope fragments of a bcl family member which possess a bcl bioactivity. Such members can be readily identified using the subject screening assays, described herein. In other embodiments “bcl family members” include polypeptides which comprise bcl domains, which confer bcl bioactivity, such as, for example, BH1, BH2, or BH4. The terms protein, polypeptide, and peptide are used interchangeably herein. Exemplary bcl family members include: bcl-2 , BCl-x_{L}, Bcl-xₛ. Bad. Bax, and others (Merry, D. B. et al. Development 120, 301-311 (1994); Núñez, G. et al. Immunol. Today 15, 582-588 (1994)). In preferred embodiments the bcl family member is a bcl-x_{L} molecule or fragment thereof In particularly preferred embodiments the bcl family member is a bcl-2 molecule or fragment thereof.

The term “modulating” is meant to include agents which either up or downregulate, the expression or bioactivity of a bcl family member in a neural cell. In preferred embodiments, a modulating agent upregulates the expression or bioactivity of a bcl family member. Agents which upregulate expression make a quantitative change in the amount of a bcl family member in a cell, while agents which upregulate the bioactivity of a bcl family member make a qualitative change in the ability of a bcl family member to perform a bcl bioactivity. Such agents can be useful therapeutically to promote axonal growth in a cell. Accordingly, the subject methods can be carried out with BCL family member modulating agents described herein, such as, nucleic acids, peptides, and peptidomimetics, or modulating agents identified in drug screens which have a BCL family member bioactivity, for example, which agonize or antagonize the effects of a BCL family member protein.

In one aspect of the invention, bcl modulating agents are nucleic acids encoding a bcl family member polypeptide which are introduced into a cell. Exemplary agents are bcl family member nucleic acids, for example in plasmids or viral vectors. As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The use of nucleic acids having a sequence that differs from a bcl family member nucleotide sequences due to degeneracy in the genetic code are also within the scope of the invention. Such nucleic acids encode functionally equivalent peptides (i.e., a peptide having a bioactivity of a bcl polypeptide) but which differ in sequence from the sequence shown in the sequence listing due to degeneracy in the genetic code. It is understood that limited modifications to the protein can be made without destroying the biological function of the bcl family member and that only a portion of the entire primary structure may be required in order to effect activity. For example, a number of amino acids are designated by more than one triplet. Codons that specify the same amino acid, or synonyms (for example, CAU and CAC each encode histidine) may result in “silent” mutations which do not affect the amino acid sequence of a bcl polypeptide. These modifications may be deliberate, such as through site-directed mutagenesis, or accidental, e.g., through mutation. Furthermore, various other modifications can be made to the bcl family member, such as the addition of carbohydrates or lipids. Furthermore, the use of homologous bcl family members, having a bcl bioactivity, from other species is also provided for.

As used herein, a bcl modulating agent can also be a nucleic acid encoding a fragment of a bcl polypeptide. A fragment refers to a nucleic acid having fewer nucleotides than the nucleotide sequence encoding the entire mature form of a bcl protein yet which encodes a polypeptide which retains some bioactivity of the full length protein. Thus, fragments of a bcl family member which retain a bcl bioactivity are included with the definition of a bcl family member. In certain embodiments fragments encode a bcl family member polypeptide of at least about 50, at least about 75, or at least about 100 amino acids. In preferred embodiments fragments encode a bcl family of at least about 150 amino acids. In more preferred embodiments fragments encode a bcl family of at least about 200 amino acids. In particularly preferred embodiments fragments encode a bcl family of at least about 239 amino acids.

Bcl protein-encoding nucleic acids can be obtained from mRNA present in any of a number of eukaryotic cells. Nucleic acids encoding bcl polypeptides of the present invention also can be obtained from genomic DNA from both adults and embryos. For example, a gene encoding a bcl protein can be cloned from either a cDNA or a genomic library in accordance with protocols described herein, as well as those generally known to persons skilled in the art. A cDNA encoding a bcl protein can be obtained by isolating total mRNA from a cell, e.g. a mammalian cell, e.g. a human cell, including embryonic cells. Double stranded cDNAs can then be prepared from the total mRNA, and subsequently inserted into a suitable plasmid or bacteriophage vector using any one of a number of known techniques. The gene encoding a bcl protein can also be cloned using established polymerase chain reaction techniques in accordance with the nucleotide sequence information provided by the invention. Alternatively, chemical synthesis of a a bcl family member gene sequence can be performed in an automatic DNA synthesizer. The bcl nucleic acid of the invention can be either DNA or RNA.

In another embodiment a a modulating agent can be a bcl family member polypeptide which can be administered directly to a neural cell, such as, conjugated to a carrier molecule. For example, certain small peptides, such as a 9 amino acid region from the HIV TAT protein can be used to efficiently transport peptides from the extracellular milieu into cells. Importantly, these peptides can serve as carriers for the introduction of very large molecules, including proteins, into mammalian cells. For example, the HIV TAT peptide can be used.

The polypeptide of this invention can be a full length protein or fragment thereof. The fragment is of a size which allows it to perform its intended function. For example, the family member polypeptide can have a length of at least about 20 amino acids, at least about 50 amino acids, at least about 75 amino acids, at least about 100 amino acids, or at least about 150 amino acids.

In other embodiments, a bcl modulating agent can be a bcl family member which has undergone posttranslational modification. For example, bcl-2 in which a putative negative regulatory loop, containing the major serine/threonine phosphorylation sites, of the protein has been deleted has been shown to have enhanced activity (Gajewski and Thompson. 1996. Cell 87:589). BCL family members which are modified to resist proteolysis may also have enhanced activity. (Strack et al. 1996. Proc. Natl. Acad. Sci. USA 93:9571).

In certain embodiments it will be advantageous to provide homologs of one of the subject BCL family member polypeptides which function in a limited capacity as one of either a BCL family member agonist (mimetic) or a BCL family member antagonist, in order to promote or inhibit only a subset of the biological activities of the naturally-occurring form of the protein. Thus, specific biological effects can be elicited by treatment with a homolog of limited function, and with fewer side effects relative to treatment with agonists or antagonists which are directed to all of the biological activities of naturally occurring forms of BCL family member proteins.

Homologs of each of the subject BCL family member proteins can be generated by mutagenesis, such as by discrete point mutation(s), or by truncation. For instance, mutation can give rise to homologs which retain substantially the same, or merely a subset, of the biological activity of the BCL family member polypeptide from which it was derived. Alternatively, antagonistic forms of the protein can be generated which are able to inhibit the function of the naturally occurring form of the protein, such as by competitively binding to a BCL family member binding protein. In addition, agonistic forms of the protein may be generated which are constituatively active. Thus, the mammalian BCL family member protein and homologs thereof provided by the subject invention may be either positive or negative regulators of axonal growth.

The recombinant BCL family member polypeptides of the present invention also include homologs of the wild type BCL family member proteins, such as versions of those proteins which are resistant to proteolytic cleavage, as for example, due to mutations which alter ubiquitination or other enzymatic targeting associated with the protein.

BCL family member polypeptides may also be chemically modified to create BCL family member derivatives by forming covalent or aggregate conjugates with other chemical moieties, such as glycosyl groups, lipids, phosphate, acetyl groups and the like. Covalent derivatives of BCL family member proteins can be prepared by linking the chemical moieties to functional groups on amino acid sidechains of the protein or at the N-terminus or at the C-terminus of the polypeptide.

Modification of the structure of the subject mammalian BCL family member polypeptides can be for such purposes as enhancing therapeutic or prophylactic efficacy, stability (e.g., ex vivo shelf life and resistance to proteolytic degradation in vivo), or post-translational modifications (e.g., to alter the phosphorylation pattern of protein). Such modified peptides, when designed to retain at least one activity of the naturally-occurring form of the protein, or to produce specific antagonists thereof, are considered functional equivalents of the BCL family member polypeptides described in more detail herein. Such modified peptides can be produced, for instance, by amino acid substitution, deletion, or addition.

For example, it is reasonable to expect that an isolated replacement of a leucine with an isoleucine or valine, an aspartate with a glutamate, a threonine with a serine, or a similar replacement of an amino acid with a structurally related amino acid (i.e. isosteric and/or isoelectric mutations) will not have a major effect on the biological activity of the resulting molecule. Conservative replacements are those that take place within a family of amino acids that are related in their side chains. Genetically encoded amino acids can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. In similar fashion, the amino acid repertoire can be grouped as (1) acidic=aspartate, glutamate; (2) basic=lysine, arginine, histidine, (3) aliphatic=glycine, alanine, valine, leucine, isoleucine, serine, threonine, with serine and threonine optionally grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 2nd ed., Ed. by L. Stryer, WH Freeman and Co.: 1981). Whether a change in the amino acid sequence of a peptide results in a functional BCL family member homolog (e.g. functional in the sense that the resulting polypeptide mimics or antagonizes the wild-type form) can be readily determined by assessing the ability of the variant peptide to produce a response in cells in a fashion similar to the wild-type protein, or to competitively inhibit such a response. Polypeptides in which more than one replacement has taken place can readily be tested in the same manner.

Full length proteins or fragments corresponding to one or more particular motifs and/or domains or to arbitrary sizes, for example, at least about 5, 10, 25, 50, 75, 100, 125, 150 amino acids in length are within the scope of the present invention. For example, isolated BCL family member polypeptides can include all or a portion of an amino acid sequence corresponding to a BCL family member polypeptide. Isolated peptidyl portions of BCL family member proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, a BCL family member polypeptide of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of a wild-type (e.g., “authentic”) BCL family member protein.

This invention further provides a method for generating sets of combinatorial mutants of the subject BCL family member proteins as well as truncation mutants, and is especially useful for identifying potential variant sequences (e.g. homologs) that modulate a BCL family member bioactivity. The purpose of screening such combinatorial libraries is to generate, for example, novel BCL family member homologs which can act as either agonists or antagonist, or alternatively, possess all together novel activities. To illustrate, combinatorially derived homologs can be generated to have an increased potency relative to a naturally occurring form of the protein.

Likewise, BCL family member homologs can be generated by the present combinatorial approach to selectively inhibit (antagonize) an authentic BCL family member. For instance, mutagenesis can provide BCL family member homologs which are able to bind other signal pathway proteins (or DNA) yet prevent propagation of the signal, e.g. the homologs can be dominant negative mutants. Moreover, manipulation of certain domains of BCL family member by the present method can provide domains more suitable for use in fusion proteins.

In one embodiment, the variegated library of BCL family member variants is generated by combinatorial mutagenesis at the nucleic acid level, and is encoded by a variegated gene library. For instance, a mixture of synthetic oligonucleotides can be enzymatically ligated into gene sequences such that the degenerate set of potential BCL family member sequences are expressible as individual polypeptides, or alternatively, as a set of larger fusion proteins (e.g. for phage display) containing the set of BCL family member sequences therein.

There are many ways by which such libraries of potential BCL family member homologs can be generated from a degenerate oligonucleotide sequence. Chemical synthesis of a degenerate gene sequence can be carried out in an automatic DNA synthesizer, and the synthetic genes then ligated into an appropriate expression vector. The purpose of a degenerate set of genes is to provide, in one mixture, all of the sequences encoding the desired set of potential BCL family member sequences. The synthesis of degenerate oligonucleotides is well known in the art (see for example, Narang, SA (1983) Tetrahedron 39:3; Itakura et al. (1981) Recombinant DNA, Proc 3rd Cleveland Sympos. Macromolecules, ed. A G Walton, Amsterdam: Elsevier pp273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

Likewise, a library of coding sequence fragments can be provided for a BCL family member clone in order to generate a variegated population of BCL family member fragments for screening and subsequent selection of bioactive fragments. A variety of techniques are known in the art for generating such libraries, including chemical synthesis. In one embodiment, a library of coding sequence fragments can be generated by (i) treating a double stranded PCR fragment of a BCL family member coding sequence with a nuclease under conditions wherein nicking occurs only about once per molecule; (ii) denaturing the double stranded DNA; (iii) renaturing the DNA to form double stranded DNA which can include sense/antisense pairs from different nicked products; (iv) removing single stranded portions from reformed duplexes by treatment with S1 nuclease; and (v) ligating the resulting fragment library into an expression vector. By this exemplary method, an expression library can be derived which codes for N-terminal, C-terminal and internal fragments of various sizes.

A wide range of techniques are known in the art for screening gene products of combinatorial libraries made by point mutations or truncation, and for screening cDNA libraries for gene products having a certain property. Such techniques will be generally adaptable for rapid screening of the gene libraries generated by the combinatorial mutagenesis of BCL family member homologs. The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate BCL family member sequences created by combinatorial mutagenesis techniques.

In one embodiment, cell based assays can be exploited to analyze the variegated BCL family member library. For instance, the library of expression vectors can be transfected into a neural cell line, preferably a neural cell line that does not express a functional BCL family member. The effect of the BCL family member mutant can be detected, e.g. axonal growth. Plasmid DNA can then be recovered from the cells which show potentiation of a BCL family member bioactivity, and the individual clones further characterized.

Combinatorial mutagenesis has the potential to generate very large libraries of mutant proteins, e.g., in the order of 10²⁶ molecules. Combinatorial libraries of this size may be technically challenging to screen even with high throughput screening assays. To overcome this problem, a new technique has been developed recently, recrusive ensemble mutagenesis (REM), which allows one to avoid the very high proportion of non-functional proteins in a random library and simply enhances the frequency of functional proteins, thus decreasing the complexity required to achieve a useful sampling of sequence space. REM is an algorithm which enhances the frequency of functional mutants in a library when an appropriate selection or screening method is employed (Arkin and Yourvan, 1992, PNAS USA 89:7811-7815; Yourvan et al., 1992, Parallel Problem Solving from Nature, 2., In Maenner and Manderick, eds., Elsevir Publishing Co., Amsterdam, pp. 401-410; Delgrave et al., 1993, Protein Engineering 6(3):327-331).

The invention also provides for reduction of the mammalian BCL family member proteins to generate mimetics, e.g. peptide or non-peptide agents. In certain embodiments such mimetics are able to disrupt binding of a mammalian BCL family member polypeptide of the present invention with BCL family members binding proteins or interactors. Thus, such mutagenic techniques as described above are also useful to map the determinants of the BCL family member proteins which participate in protein-protein interactions involved in, for example, binding of the subject mammalian BCL family member polypeptide to proteins which may function upstream (including both activators and repressors of its activity) or to proteins or nucleic acids which may function downstream of the BCL family member polypeptide, whether they are positively or negatively regulated by it. To illustrate, the critical residues of a subject BCL family member polypeptide which are involved in molecular recognition of interactor proteins upstream or downstream of a BCL family member (such as, for example BH1 domains, BH2 domains) can be determined and used to generate BCL family member-derived peptidomimetics which competitively inhibit binding of the authentic BCL family member protein to that moiety. By employing, for example, scanning mutagenesis to map the amino acid residues of each of the subject BCL family member proteins which are involved in binding other extracellular proteins, peptidomimetic modulating agents can be generated which mimic those residues of the BCL family member protein which facilitate the interaction. Such mimetics may then be used to interfere with the normal function of a BCL family member protein. For instance, non-hydrolyzable peptide analogs of such residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted g lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), b-turn dipeptide cores (Nagai et al. (1985) Tetrahedron Lett 26:647; and Sato et al. (1986) J Chem Soc Perkin Trans 1:1231), and b-aminoalcohols (Gordon et al. (1985) Biochem Biophys Res Commun126:419; and Dann et al. (1986) Biochem Biophys Res Commun 134:71).

Other exemplary bcl modulating agents include any compounds which, when contacted with a cell, alter the “bioactivity” of a bcl family member protein. For example, the bioactivity of a bcl family member can be increased by turning on a bcl family member gene and increasing its transcription, stabilizing a bcl family member mRNA, increasing the rate of bcl family member protein synthesis, decreasing the rate of bcl family member protein degradation, animating bcl family member functions, helping proper folding of a bcl family member protein, aiding a bcl family member protein in reaching its subcellular compartment(s), promoting bcl family member interactions with relevant targets, such as for example Raf-1 (Wang et al. 1996 Cell 87:629), and/or activating directly or indirectly targets downstream of a bcl family member.

The term “bioactivity” of a bcl family member is meant to include the ability of a molecule to promote axonal growth. Increases in the bioactivity of a bcl family member can occur absent any alteration in transcription of a bcl family member. For example, bioactivity can be altered by allosteric molecules which bind to or interact with a bcl family member. Bioactivity of a bcl family member can also be assessed by its ability to compete with a bcl-2 molecule in its ability to promote axonal growth. Competition with a bcl-2 molecule can be tested, for example in cells which express bcl-2 and a bcl family member and inhibition of axonal growth can be quantitated.

Still other bcl modulating agents are molecules which influence the bioactivity of a bcl family member protein indirectly, by modulating molecules which bind to a bcl family member in order to effect changes in the bioactivity of a bcl family member. Exemplary agents which bind to and alter the bioactivity of bcl family members include Bax, Bak, Mel-1, Bag, Nip1, Nip2, and Nip 3 (Farrow and Brown Curr Opin in Genetics and Devo. 6:45(1996)). For example, Raf-1 has also been found to interact with bcl-2 (Gajewski and Thompson. 1996. Cell 87:589). Therefore, the present invention also provides for modulating bcl family members by modulating proteins which interact with and affect the bioactivity of a bcl family member, such as by changing the ratio between a bcl family member and proteins with which they interact.

In yet another embodiment, this invention also teaches methods to screen for pharmacologically acceptable agents that can reach the CNS and turn on a bcl family member gene, stabilize bcl family member mRNA, increase rate of bcl family member protein synthesis, decrease bcl family member protein degradation, enhance bcl family member bioactivity, animate bcl family member functions, help proper folding of bcl family member protein, aid bcl family member protein to reach its subcellular compartment(s), promote bcl family member interactions with relevant targets, such as Raf-1 at mitochondria (Wang et al. 1996 Cell 87:629), and/or activate directly or indirectly targets downstream of a bcl family member.

Neurons cultured in Terasaki plates, 96-well plates, and recently developed 864-well plates may be used for screenings of a larger number of agents for any or all of biological activities listed above. Agents appropriate for such screenings include any of the 21-million structures listed in Chemical Abstract Database, any natural products, large or small, derived from animals, plants, microorganisms, marine organisms, insects, fermentation or biotransformation, or any future molecules to be generated by conventional organic synthesis, rational drug design or combinatorial chemistry. Robotic high-throughput and ultrahigh-throughput screening methods may be employed to identify such pharmacological agents with desirable activities that promote CNS regeneration via a bcl family member pathway.

Assay endpoints for robotic screenings include, but are not limited to, increased expression of a bcl family member (by immunofluoresence or immunoperoxidase with antibodies specific for bcl family member protein), increased mitochondrial membrane potentials (a consequence of increased bcl family member expression that can be detected by fluorescent, delocalized lipophilic cations), resistance to uncouplers for oxidative phosphorylation such as dinitrophenols or FCCP (a consequence of increased bcl family member expression that can be monitored by fluorescent dyes), resistance to apoptosis inducers (a consequence of increased bcl family member expression measurable by MTT or MTS dyes), and/or increased neural regeneration and neurite outgrowth.

Active compounds revealed by the assays listed above shall be further characterized by comparing their effects on neurons derived from uncompromised mice, bcl family member (−/−) knockout mice, or bcl family member transgenic mice. Pharmacological agents that promote neural regeneration via a bcl family member or its mRNA or its protein should be inactive in bcl-2 family member (−/−) knockout mice. Agents that turn on a bcl family member gene should be active in neurons derived from uncompromised mice. Agents that stabilize bcl family member mRNA or proteins should be active in neurons derived from bcl family member transgenic mice. Pharmacological agents that animate bcl family member function or activate targets downstream of bcl family member may still be active in bcl family member (−/−) knockout mice.

Thus, this invention embodies any screening methods that allow the identification of any molecules, large or small, naturally occurring or man-made (by conventional organic synthesis or combinatorial chemistry), that act on bcl family member pathway in neurons, be it at bcl family member gene or its mRNA or its protein, or at bcl family member protein's downstream targets, and are able to induce their regeneration.

In other embodiments of the invention, members of the bcl family which can function to promote axonal growth can be identified in axonal growth screening assays (AGSAs). In the subject AGSAs, first a tissue sample, which contains the source of axons, is placed in contact with a second tissue sample into which said axons can grow. The expression of a bcl family member can be modulated in the first tissue sample and the effects thus can be selected on axonal growth can be determined. Thus, bcl family members can be selected which have a bcl bioactivity, e.g., promote axonal growth. Axonal growth can be measured by determining or quantifying the extension of axon(s), for example, as described in the appended Exemplification.

The subject AGSAs can also be used to select agents which can modulate axonal growth by providing a first tissue sample which contains axons and abutting it with a second tissue sample into which said axons can grow. Various agents can then be tested for effects on axonal growth by addition of the agents to the culture and agents which promote axonal growth can be selected. Such agents may be obtained, for example, through rational design or random drug-screening.

The modulation of bcl family member bioactivity can occur either in vitro or in vivo.

In one embodiment a bcl family member can be modulated in a neural cell in vitro. Bcl modulation can be tested by measuring a bcl bioactivity in the cells (i.e., the promotion of axonal growth) or by performing immunoblot analysis, immunoprecipitation, or ELISA assays. The neural cell can be transplanted into a subject who has suffered a traumatic injury or with a state characterized by diminished axonal growth.

As used herein, the term “state characterized by diminished potential for axonal growth” is meant to encompass a state or disorder which would benefit from the axonal growth induced by increased expression of a bcl family member. Reduced expression of a bcl family member may occur normally, as in adult neurons of the CNS, or because of a pathologic condition brought about by the misexpression of a bcl family member. “Diminished” as used herein is meant to include states in which axonal growth is absent as well those in which it is reduced.

The present invention specifically provides for applications of the method of this invention in the treatment of states characterized by diminished potential for axonal growth. Exemplary states “characterized by diminished potential for axonal growth” include neurological conditions derived from injuries of the spinal cord or compression of the spinal cord, or complete or partial transection of the spinal cord. For example, injuries may be caused by: (i) acute, subacute, or chronic injury to the nervous system, including traumatic injury (e.g. severing or crushing of a neuron(s)), such as that brought about by an automobile accident, fall, or knife or bullet wound, (ii) chemical injury, (iii) vascular injury or blockage, (iii) infectious or inflammatory injury such as that caused by a condition known as transverse myelitis, or (iii) a tumor-induced injury, whether primary or metastatic. Thus, injuries leading to a state associated with diminished potential for axonal growth can be direct, e.g., due to concussion, laceration, or intramedullary hemorrhage, or indirect, e.g., due to extramedullary pressure of loss of blood supply and infarction.

The present invention will be useful in treating neurons in both the descending (e.g., corticospinal tract) and ascending tracts (e.g., the dorsal column-medial lemniscal system, the lateral spinothalamic tract, and the spinocerebellar tract) of the spinal cord and in the reestablishment of appropriate spinal connections.

Common mechanisms of spinal cord injury include fractures of the vertebrae, which can damage the spinal cord from the concussive effect of injury due to displaced bony fragments, or damaged blood vessels, or contusion of emerging nerve roots. Dislocation of vertebrae can also cause spinal cord damage; dislocation is often the result of the rupture of an intervertebral disk, and may result in partial or complete severance of the spinal cord. Penetrating wounds can also cause severance, or partial severance of the cord. Epidural hemorrhage and spinal subdural hematoma can result in progressive paraparesis due to pressure on the spinal cord. Examples of indirect injury to the spinal cord include damage induced by a blow on the head or a fall on the feet. Intramedullary injury can be the result of direct pressure on the cord or the passage of a pressure wave through the cord, laceration of the cord by bone, or the rupture of a blood vessel during the passage of a pressure wave through the cord with a hemorrhage into the cord. Intramedullary bleeding and hematoma formation can also be caused by rupture of a weakened blood vessel. Ischemic damage can occur following compression of the anterior spinal artery, pressure on the anastomotic arteries, or damage to major vessels (Gilroy, in Basic Neurology McGraw-Hill, Inc. New York, N.Y. (1990). The present invention will also be useful in promoting the recovery of subjects with a herniated disks, hyperextension-flexion injuries to the cervical spine and cervical cord, and cervical spondylosis.

In addition to treating movement disorders, the present invention will be useful in treating disorders of the brain, e.g. the brain stem and in enhancing brain or brain stem function in a subject with a state characterized by diminished potential for axonal growth. For example, the present invention can be used in the treatment of brain damage. For example, the brain damage can be caused by stroke, bleeding trauma, or can be tumor-related brain damage.

The present invention will also be useful in treating peripheral neuropathies. Damage to peripheral nerves can be temporary or permanent and, accordingly, the present invention can hasten recovery or ameliorate symptoms. Peripheral neuropathies include, among others, those caused by trauma, diabetes mellitus, infarction of peripheral nerves, herniated disks, epidural masses, and postinfectious (or postvaccinal) polyneurites. The symptoms of peripheral neuropathies which will benefit from the instant invention include muscle wasting and weakness, atrophy, the appearance of fasciculations, impaired tendon reflexes, impaired sensation, dysethesias or paresthesias, loss of sweating, alteration in bladder function, constipation, causalgia, and male impotence.

The use of the instant invention to treat neurodegenerative diseases which will benefit by enhanced axonal growth is also provided for. In preferred embodiments the subject invention is used to treat neurodegenerative diseases, such as, Pick's disease, progressive aphasia without dementia, supranuclear palsy, Shy-Drager Syndrome, Friedreich's ataxis, olivopontocerebellar degeneration, vitamin E deficiency and spinocerebellar degeneration, Roussy-Levy Syndrome, and hereditary Spastic ataxia or paraparesis. In addition, treatment of other disorders of the spinal cord, such as amyotrophic lateral sclerosis, spinal muscular atrophies, and multiple sclerosis are intended to be part of the present invention. In other embodiments the present invention will be useful in ameliorating the symptoms of neural degeneration such as that induced by vitamin B12 deficiency, or associated with HIV infection (AIDS), or HTLV-1 infection. In particularly preferred embodiments of the present invention are used to treat any neurodegenerative disorder with the exception of Alzheimer's disease, Parkinson's disease, cancer, or viral infections. The anti-apoptotic treatment of Alzheimer's disease, Parkinson's disease, cancer, or viral infection are intended to be part of this invention.

Other states characterized by diminished potential for axonal growth which will benefit by the present invention will be apparent to one of ordinary skill in the art.

The term “treatment” is intended to include prevention and/or reduction in the severity of at least one symptom associated with the state being treated. The term also is intended to include enhancement of the subject's recovery from the state.

The term “subject” as used herein is meant to encompass mammals. As such the invention is useful for the treatment of domesticated animals, livestock, zoo animals, etc. Examples of subjects include humans, cows, cats, dogs, goats, and mice. In preferred embodiments the present invention is used to treat human subjects.

The present invention provides for the additional administration of agents which create an “environment” favorable to axonal growth. Exemplary agents include trophic factors, receptors, extracellular matrix proteins, intrinsic factors, or adhesion molecules. Exemplary trophic factors include NGF, BDNF, NT-3, 4, 5, or 6, CNTF, LIF, IGFI, IGFII, GDNF, GPA, bFGF, TGFβ, and apolipoprotein E. Exemplary receptors include the Trk family of receptors. An exemplary extracellular matrix protein is laminin. Exemplary intrinsic factors include GAP-43 (also known as B50, pp46, neuromodulin, and F1) and ameloid precursor protein (APP) (Moya et al. Dev. Biol. 161:597 (1994)). Exemplary adhesion molecules include NCAM and L1. Nucleic acids encoding these polypeptides, or the polypeptides may be used. The use of peptide fragments of any of the above axonal growth enhancers could also be used.

In another embodiment the invention provides a method of treating a subject that has suffered a traumatic injury in which nerve cell injury has occurred, in which a subject is treated with a bcl modulating agent, e.g., such that axonal growth occurs. Exemplary traumatic injuries include severing or crushing of a neuron(s), such as that brought about by an automobile accident, fall, or knife or bullet wound, as well as others described herein.

The present invention also provides a method of treating a subject for a state characterized by diminished potential for axonal growth by administering a therapeutically effective amount of an agent which modulates the bioactivity or expression of a bcl family member in a subject.

This invention also provides means for delivery of a bcl modulating agents to a neural cell. In certain embodiments gene constructs containing nucleic acid encoding a bcl family member are provided. As used herein the term “gene construct” is meant to refer to a nucleic acid encoding a bcl family member which is capable of being heterologously expressed in a neural cell. In certain embodiments, the a bcl family member may be operably linked to at least one transcriptional regulatory sequence for the treatment of a state characterized by diminished potential for axonal growth. Operably linked is intended to mean that the nucleotide sequence is linked to a regulatory sequence in a manner which allows expression of the nucleotide sequence. Regulatory sequences are art-recognized and are selected to direct expression of the subject bcl proteins. Accordingly, the term transcriptional regulatory sequence includes promoters, enhancers and other expression control elements. Such regulatory sequences are described in Goeddel; Gene Expression Technology: Methods in Enzymology 185, Academic Press, San Diego, Calif. (1990). For instance, any of a wide variety of expression control sequences-sequences that control the expression of a DNA sequence when operatively linked to it may be used in these vectors to express DNA sequences encoding the bcl polypeptides of this invention. Such useful expression control sequences, include, for example, a viral LTR, such as the LTR of the Moloney murine leukemia virus, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage 1, the control regions for fd coat protein, the promoter for 3-phosphoglycerate kinase or other glycolytic enzymes, the promoters of acid phosphatase, e.g., Pho5, the promoters of the yeast a-mating factors, the polyhedron promoter of the baculovirus system and other sequences known to control the expression of genes of prokaryotic or eukaryotic cells or their viruses, and various combinations thereof. In preferred embodiments the promoter is designed specifically for expression in neural cells. In particularly preferred embodiments the promoter is a neural specific enolase promoter. It should be understood that the design of the expression vector may depend on such factors as the choice of the host cell to be transformed and/or the type of protein desired to be expressed. Moreover, the vector's copy number, the ability to control that copy number and the expression of any other proteins encoded by the vector, such as markers, should also be considered.

In certain embodiments it will be desirable to additionally administer agents which create an environment favorable to axonal growth into an expression vector comprising a nucleic acid encoding a bcl family member. Examples of classes of such agents include trophic factors, receptors, extracellular matrix proteins, or intrinsic factors. Exemplary trophic factors include NGF, BDNF, NT-3, 4, 5, or 6, CNTF, LIF, IGFI, IGFII, GDNF, GPA, bFGF, TGFb, and apolipoprotein E. Exemplary receptors include the Trk family of receptors. An exemplary extracellular matrix protein is laminin. Exemplary intrinsic factors include GAP-43 and ameloid precursor protein (APP)(Moya et al. Dev. Biol. 161:597 (1994)). Exemplary adhesion molecules include NCAM and L1.

Agents which provide an environment favorable to axonal growth can be administered by a variety of means. In certain embodiments they can be incorporated into the gene construct. In other embodiments, they may be injected, either locally or systemically. In other embodiments such agents can be supplied in conjunction with nerve guidance channels as described in U.S. Pat. Nos. 5,092,871 and 4,955,892. Accordingly, a severed axonal process can be directed toward the nerve ending from which it was severed by a prosthesis nerve guide which contains a non-bcl agent as, e.g. a semi-solid formulation, or which is derivatized along the inner walls of the nerve guidance channel. These agents may be adminestered simultaneously with a bcl modulating agent, or not.

In certain embodiments of the invention, for example in the treatment of longstanding injury (e.g., when there has been significant colateral sprouting of a neural cell) it may be desirable to combine treatment with the subject bcl modulating agents with a “pruning procedure” to remove rostral sprouting (Schneider, G. E. Brain. Bahav. Evol. 8:73 (1973)).

Expression constructs of the subject bcl modulating agents, may be administered in a biologically effective carrier, e.g. any formulation or composition capable of effectively delivering the bcl gene to cells in vivo. Approaches include insertion of the subject gene in viral vectors including recombinant retroviruses, adenovirus, adeno-associated virus, and herpes simplex virus-1, or other attenuated viruses, or recombinant bacterial or eukaryotic plasmids which can be taken up by the damaged axon. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO₄ precipitation carried out in vivo. It will be appreciated that the choice of the particular gene delivery system will depend on such factors as the intended target and the route of administration, e.g. locally or systemically. In particularly preferred embodiments, the constructs employed are specially formulated to cross the blood brain barrier. Furthermore, it will be recognized that the gene constructs provided for in vivo modulation of bcl expression are also useful for in vitro modulation of bcl expression in cells, such as for use in the ex vivo assay systems described herein.

A preferred approach for in vivo introduction of nucleic acid into a cell is by use of a viral vector containing nucleic acid, e.g. a DNA, encoding the particular form of the bcl polypeptide desired. Infection of cells with a viral vector has the advantage that a large proportion of the targeted cells can receive the nucleic acid. Additionally, molecules encoded within the viral vector, e.g., by aDNA contained in the viral vector, are expressed efficiently in cells which have taken up viral vector nucleic acid.

Retrovirus vectors and adeno-associated virus vectors can be used as the gene delivery system of the present invention for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. Blood 76:271(1990). Thus, recombinant retrovirus can be constructed in which part of the retroviral coding sequence (gag, pol, env) has been replaced by nucleic acid encoding one of the subject receptors rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of retroviruses include pLJ, pZIP, pWE and pEM which are well known to those skilled in the art. Examples of packaging virus lines for preparing both ecotropic and amphotropic retroviral systems include ψCrip, ψCre, ψ2 and ψAm. Retroviruses have been used to introduce a variety of genes into many different cell types in vitro and/or in vivo (see for example Eglitis, et al. Science 230:1395-1398(1985); Danos and Mulligan Proc. Natl. Acad. Sci USA 85:6460-6464(1988); Wilson et al. Proc. Natl. Acad. Sci. USA 85:3014-3018(1988); Armentano et al. Proc. Natl. Acad. Sci USA 87:6141-6145(1990); Huber et al. Proc. Natl. Acad. Sci USA 88:8039-8043(1991); Ferry et al. Proc. Natl. Acad. Sci. USA 88:8377-8381(1991); Chowdhury et al. Science 254:1802-1805(1991); van Beusechem et al. Proc. Natl. Acad. Sci USA 89:7640-7644(1992); Kay et al. Human Gene Therapy 3:641-647(1992); Dai et al. Proc. Natl. Acad Sci. USA 89:10892-10895(1992); Hwu et al. J. Immunol. 150:4104-4115(1993); U.S. Pat. Nos. 4,868,116; 4,980,286; PCT Application WO 89/07136; PCT Application WO 89/02468; PCT Application WO 89/05345; and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications WO93/25234 and WO94/06920). For instance, strategies for the modification of the infection spectrum of retroviral vectors include: coupling antibodies specific for cell surface antigens to the viral env protein (Roux et al. PNAS 86:9079-9083(1989); Julan et al. J. Gen Virol 73:3251-3255(1992); and Goud et al. Virology 163:251-254(1983)); or coupling cell surface receptor ligands to the viral env proteins (Neda et al. J. Biol Chem 266:14143-14146(1991)). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, can also be used to convert an ecotropic vector in to an amphotropic vector.

Moreover, use of retroviral gene delivery can be further enhanced by the use of tissue- or cell-specific transcriptional regulatory sequences which control expression of the bcl gene of the retroviral vector.

Another viral gene delivery system useful in the present invention utilitizes adenovirus-derived vectors. The genome of an adenovirus can be manipulated such that it encodes and expresses a gene product of interest but is inactivated in terms of its ability to replicate in a normal lytic viral life cycle. See for example Berkner et al. BioTechniques 6:616(1988); Rosenfeld et al. Science 252:431-434(1991); and Rosenfeld et al. Cell 68:143-155(1992). Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses can be advantageous in certain circumstances in that they are not capable of infecting nondividing cells and can be used to infect a wide variety of cell types (Rosenfeld et al. supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham J. Virol. 57:267(1986)). Most replication-defective adenoviral vectors currently in use and therefore favored by the present invention are deleted for all or parts of the viral E1 and E3 genes but retain as much as 80% of the adenoviral genetic material (see, e.g., Jones et al. Cell 16:683(1979); Berkner et al., supra; and Graham et al. in Methods in Molecular Biology, E. J. Murray, Ed. (Humana, Clifton, N.J., 1991) vol. 7. pp. 109-127). Expression of the inserted bcl gene can be under control of, for example, the E1A promoter, the major late promoter (MLP) and associated leader sequences, the E3 promoter, or exogenously added promoter sequences.

Yet another viral vector system useful for delivery of the subject bcl gene is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. Curr. Topics in Micro. and Immunol. 158:97-129(1992)). It is also one of the few viruses that may integrate its DNA into non-dividing cells, and exhibits a high frequency of stable integration (see for example Flotte et al. Am. J. Respir. Cell. Mol. Biol. 7:349-356(1992); Samulski et al. J. Virol. 63:3822-3828(1989); and McLaughlin et al. J. Virol. 62:1963-1973 (1989)). Vectors containing as little as 300 base pairs of AAV can be packaged and can integrate. Space for exogenous DNA is limited to about 4.5 kb. An AAV vector such as that described in Tratschin et al. Mol. Cell. Biol. 5:3251-3260 (1985) can be used to introduce DNA into cells. A variety of nucleic acids have been introduced into different cell types using AAV vectors (see for example Hermonat et al. Proc. Natl. Acad. Sci. USA 81:6466-6470(1984); Tratschin et al. Mol. Cell. Biol. 4:2072-2081(1985); Wondisford et al. Mol. Endocrinol. 2:3239(1988); Tratschin et al. J Virol. 51:611-619 (1984); and Flotte et al. J. Biol. Chem. 268:3781-3790(1993)).

Replication defective Herpes simplex virus-1 (HSV-1) vectors have been shown to achieve efficient transduction and expression of heterologous genes in the nervous system (Dobson et al. Neuron. 5:353(1990); Federoff et al. Proc. Natl Acad. Sci. U.S.A. 89:1636(1992); Andersen et al. Hum Gene Ther. 3:487(1992); Huang et al. Exp Neurol. 115:303(1992); Fink et al. Hum Gene Ther. 3:11(1992); Breakefield et al. in Gene Transfer and Therapy in the Nervous System. Heidelberg, FRG: Springer-Verlagpp 45-48(1992); and Ho et al. Proc Natl. Acad. Sci U.S.A. 90:3655(1993)). HSV-2 vectors expressing bcl have also been described (Linnik et al. Stroke. 26:1670(1995); Lawrence et al. J. Neuroscience. 16:486(1996)).

In addition to viral transfer methods, such as those illustrated above, non-viral methods can also be employed to cause expression of a bcl polypeptide in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject bcl polypeptide gene by the targeted cell. Exemplary gene delivery systems of this type include liposomal derived systems, poly-lysine conjugates, and artificial viral envelopes.

In a representative embodiment, a gene encoding the subject bcl polypeptides can be entrapped in liposomes bearing positive charges on their surface (e.g., lipofectins) and (optionally) which are tagged with antibodies against cell surface antigens of the target tissue (Mizuno et al. (1992) No Shinkei Geka 20:547-551; PCT publication WO91/06309; Japanese patent application 1047381; and European patent publication EP-A-43075). For example, lipofection of cells can be carried out using liposomes tagged with monoclonal antibodies against any cell surface antigen present on the target cells.

In one aspect, the invention features a pharmaceutical preparation which includes a recombinant transfection system. The term “recombinant transfection system” is meant to include a gene construct including a nucleic acid encoding a bcl modulating agent, a gene delivery composition, and, optionally one or more non-bcl agents as described herein, which create an environment favorable to axonal growth. Such “gene delivery compositions” are capable of delivering a nucleic acid encoding a bcl family member to its intended target, e.g., a neural cell and can include the compositions described herein, such as, a viral vector or recombinant bacterial or eukaryotic plasmids. Plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO₄ precipitation.

In clinical settings, the gene delivery systems for the therapeutic bcl gene can be introduced into a subject by a number of methods, each of which is art-recognized. For instance, a pharmaceutical preparation of the gene delivery system can be introduced systemically, e.g. by intravenous injection, and specific transduction of the nucleic acid in the target cells occurs predominantly from specificity of transfection provided by the gene delivery composition, site of administration, cell-type or tissue-type expression due to the transcriptional regulatory sequences controlling expression of the receptor gene, or a combination thereof. In other embodiments, initial delivery of the recombinant gene is more limited with introduction into the animal being quite localized, for example delivery can be targeted to a specific area of the brain, e.g., the injection can be intraventricular. To facilitate local delivery, the gene delivery vehicle can be introduced by stereotactic injection (e.g. Chen et al. PNAS 91: 3054-3057(1994) ).

The pharmaceutical preparation of the gene delivery composition can contain the gene delivery system in an acceptable diluent, or can contain a slow release matrix in which the gene delivery vehicle is imbedded. Alternatively, where the complete gene delivery system can be produced intact from recombinant cells, e.g. retroviral vectors, the pharmaceutical preparation can comprise one or more cells which produce the gene delivery system.

Pharmaceutical compositions containing a bcl family member polypeptide and a pharmaceutically acceptable carrier formulated for promoting axonal growth also are intended to be part of this invention. The compositions can contain the full length protein or the fragments described above. The pharmaceutical compositions containing the polypeptide can be formulated to target a neural cell, or can be specially formulated for an anti-apoptosis use such as those described herein. For example, the peptide can be conjugated for example, to a carrier or encapsulated within a delivery system.

Pharmaceutical compositions for use in accordance with the present invention may be formulated in a conventional manner using one or more physiologically acceptable carriers or excipients. Thus, the compounds and their physiologically acceptable salts and solvates may be formulated for administration, for example, by injection.

For example, the compositions of the invention can be formulated for a variety of loads of administration, including systemic. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compositions of the invention can be formulated in liquid solutions, preferably in physiologically compatible buffers such as Hank's solution or Ringer's solution. In addition, the oligomers may be formulated in solid form and redissolved or suspended immediately prior to use. Lyophilized forms are also included.

The compositions may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulation agents such as suspending, stabilizing and/or dispersing agents. Alternatively, the active ingredient may be in powder form for constitution with a suitable vehicle, e.g., sterile pyrogen-free water, or saline before use.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosages for use in humans. For example, the dosage of such compositions lies preferably within a range that includes the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma or local tissue concentration range that includes the IC₅₀ (i.e., the concentration of the test compound which achieves a half-maximal therapeutic effect, e.g., inhibition of symptoms) as determined in cell culture. Such information can be used to more accurately determine useful doses in humans. Levels in plasma or local tissue may be measured, for example, by high performance liquid chromatography.

The regimen of administration can also affect what constitutes an effective amount. The compositions of the present invention can be administered in several divided dosages, as well as staggered dosages, can be administered daily or sequentially, or the dose can be continuously infused, or can be a bolus injection. Further, the dosages of the agent(s) can be proportionally increased or decreased as indicated by the exigencies of the therapeutic or prophylactic situation.

Another embodiment of the present invention provides for a packaged drug for the treatment of a state associated with diminished potential for axonal growth, which includes a bcl modulating agent packaged with instructions for treating a subject. The “packaged drug” of the present invention can include any of the compositions described herein. The term “instructions” as used herein is meant to include the indication that the packaged drug is useful for treating a state associated with diminished potential for axonal growth and optionally may include the steps which one of ordinary skill in the art would perform to treat a subject with such a state.

Exemplification

The invention, now being generally described, will be more readily understood by reference to the following examples, which are included merely for purposes of illustration of certain aspects and embodiments of the present invention and are not intended to limit the invention. The animal models used throughout the Examples are accepted animal models and the demonstration of efficacy in these animal models is predictive of efficacy in humans.

### Experimental Methods Used in the Examples

Retinotectal Cocultures

Brains were dissected into ice-cold Gey's balanced salt solution enriched with glucose. Coronal slices through the superior colliculus were cut with a McIIwain tissue chopper at a thickness of 300 m. Retinal explants were abutted against tectal slices. Tissues were placed on the microporous membrane of Millicell wells (Millipore) and maintained in NeuralBasal medium supplemented with B27 (GIBCO Inc., New York) at 37° C. for five days. To exclude the possibility that tectal tissues from mutant mice may affect axonal growth of RGCs, a series of parallel experiments were performed in which one retinal explant of each mouse was confronted with the tectum from the same mouse, while the second retinal explant was placed against the tectum from another mouse. With this arrangement, retinal explants from each animal had the possibility of being cocultured with the tectum from a wild-type, heterozygous, or homozygous animal. The number of regenerating axons was sampled by applying the lipophilic carbocyanine fluorescent label, DiI, in crystals to fixed retinal explants. The cocultures were stored in fixative for two-four weeks to allow diffusion of the dye, and labeled retinal axons were viewed with a fluorescence microscope (Nikon).

Mouse pups were obtained from matings of males heterozygous for the bcl-2 transgene with C57BL/6J females. Four days after birth (P4), pups received a unilateral transection of the optic tract at the mid-tectal level. Regeneration of the optic tract was assessed using anterograde tracing with CT-B (cholera toxin B), ten days after nerve transection. To visualize the axons, a diaminobenzidine (DAB) color reaction was carried out using a slightly modified version of the protocol of Angelucci, et al (Angelucci, A., Clascá, F. & Sur, M. J. Neurosci. Meth. 65, 101-112 (1996)). In brief, brains were cut into 50 m sagittal sections; every other section of the brain was collected for cresyl violet staining, and the other section was incubated with primary antibody against CT-B at 4C. for 96 hr and then further processed with ABC elite kit (Vector). The brain sections were visualized with a Nikon microscope and site of the lesion was reconstructed in 3 dimensions with MIT Neurotrace computer software.

Primary cultures of dissociated retinal cells were prepared from P2 wild-type or transgenic animals. RGCs were prelabeled by injecting DiI solution (25% in Dimethyl Formamide) into the tectal region bilaterally in P0 pups. Cells were plated in 24-cell wells treated with poly-L-lysine (10 g/ml, 4° C. overnight) and coated with Human Merosin (0.2 g/ml, r.t., 2 hr)(Meyer-Franke, A. and Barres, B. A. Neuron 15, 805-819 (1995)). Cultures were maintained for 2 to 3 days in NeuralBasal medium supplemented with B27. Trypan blue staining was used to examine the viability of retinal ganglion cells (RGCs). Retinotectal cocultures prepared from wild-type P2 mice were described previously and ZVAD (Z-Val-Ala-Asp-CH₂F, Enzyme Systems Products) was added to the culture medium at the time of plating. Cell death was detected by staining with the fluorescent dye, SYTOX green fluorescent dead cell stain (Molecular Probes). Cultures were visualized under an inverted Nikon microscope equipped with Nomarski and epifluorescence illumination.

Immunoflorescent Staining

For immunofluorescence staining of bcl-2 , embryonic day 16 or 18 (E16 or E18) embryos were obtained by Caesarian section of timed mated wild-type mothers. Brains were removed and fixed in 4% paraformaldehyde overnight and cut into transverse sections of 10 μm thickness with a cryostat. Sections were blocked with PBS containing 2.5% normal goat serum, 2.5% fetal bovine albumin, and 0.3% Triton X-100 for 30 min. at room temperature, and then incubated with affinity purified primary antibody (hamster anti-mouse bcl-2, 1:50, PharMingen) at 4° C. overnight. Secondary antibody (FITC-conjugated goat antibody to hamster immunoglobulin, 1:200) was then applied to the slide for 2 hr at room temperature. The slides were rinsed several times in PBS, mounted in Fluoremount G and viewed with the fluorescence microscope.

### EXAMPLE 1

### Growth of Retinal Axons

To examine the growth of CNS axons of mice, an organotypic coculture model of the retinotectal system was established, in which the growth pattern of retinal axons closely mimics that seen in vivo (Chen, D. F., Jhaveri, S. & Schneider, G. E. Proc. Natl. Acad. Sci USA 92, 7287-7291 (1995)). Tissues from retinae and midbrain tecta of C57BL/6J mice are abutted in a culture well. Quantitative analysis of axonal growth from retinae is achieved by the standard placement of DiI into retinal explants. Cocultures prepared from animals aged embryonic day 14 (E14, day of mating=E0) through E16 were examined. Growth of retinal axons into the tectal slice was extensive (n=20); axons for E16 retinae could be observed growing into the entire tectal explant, and the number of labeled axons invading tectal tissue averaged 126±10.0. In contrast, retinal explants (n=60) prepared from animals at age E18 and older exhibited markedly reduced axonal growth. For E18 tissues, the mean result was averaging 15.5±3.3 fibers per tectal slice, while no obvious increase in cell death was observed in these cultures. This indicated that starting at E18 in mice, most RGC axons display a regenerative failure in culture. Thus, the level of expression of bcl-2 in RGCs correlates with the growth ability of retinal axons. This finding matched the previous report on the Syrian hamster (Chen et al. supra).

Previous work showed that embryonic RGCs can grow axons into tectal tissue of any age, whereas older retinae fail to grow many axons into CNS tissue of any age including into embryonic tecta. To determine which genes might play such roles in regulating the growth of retinal axons, the level of expression of several molecules, including bcl-2 , was compared with the use of immunofluorescence staining. High expression of bcl-2 at E16 in the RGC layer of retinae was found. At E18, in parallel with the onset of regenerative failure in culture, the expression of bcl-2 decreased to an undetectable level .

### EXAMPLE 2

A bcl Family Member is Required for the Growth of Axons

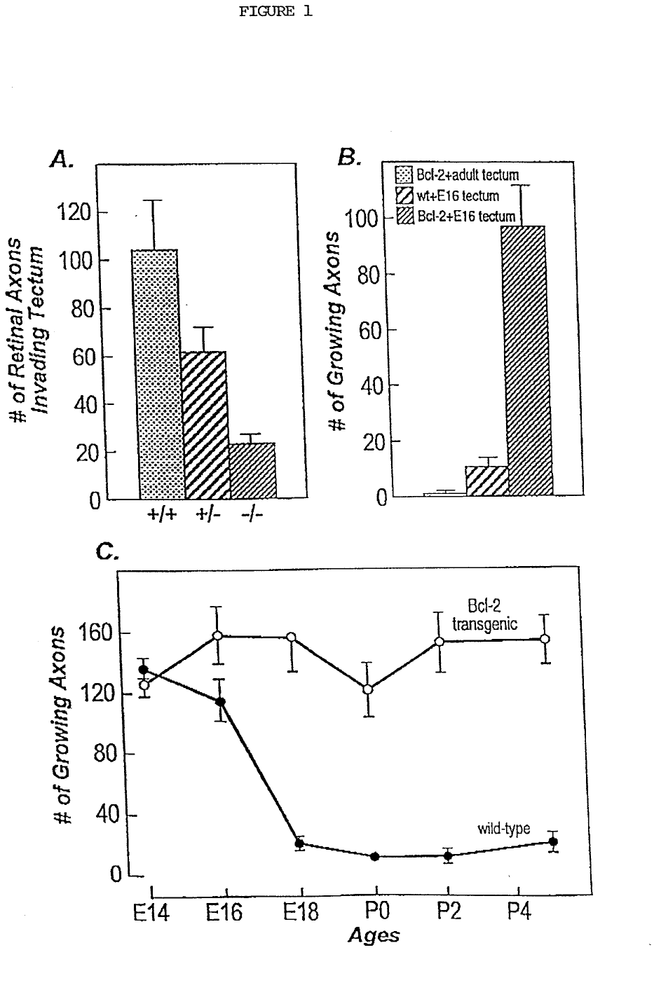

To determine whether bcl-2 is required for the growth of retinal axons, a loss-of-function animal model—mice genetically deficient in bcl-2 was studied (Veis, D. J., Sorenson, C. M., Shutter, J. R. & Korsmeyer, S. J. Cell 75, 229-240 (1993)). These mice were derived from matings of heterozygous offspring. Resulting litters contained wild-type, heterozygous, and bcl-2-deficient mice. Cocultures were prepared from E15 embryos. At this stage, retinal explants of wild-type animals showed robust neurite outgrowth. To exclude the possibility that tectal tissues from mutant mice may affect axonal growth of RGCs, a series of parallel experiments was performed in which retinal explants from each animal had the possibility of being cocultured with the tectum from a wild-type, heterozygous, or homozygous animal. Regardless of the origin of tectal tissue, retinal explants derived from embryos of heterozygous and homozygous bcl-2 mutation grew significantly fewer neurites than those from wild-type littermates (P<0.001). The numbers of labeled retinal axons were reduced by 50% in retinae prepared from heterozygous animals (62±8, n=20) and by 80% in those from homozygous animals (22±4, n=7) (FIG. 1A). There was no significant difference between groups of retinae cocultured with tecta from wild-type and mutant mice. It should be noted that the numbers of retinal axons from cultures of mice containing the homozygous bcl-2 mutation were equivalent to those of wild-type mice on E18—when most RGCs failed to grow axons into tectum.

### EXAMPLE 3

### Expression of a bcl Family Member Allowed Axon Regeneration in Adult Nerual Tissue

Since loss of bcl-2 function represses axonal growth, whether or not overexpression of bcl-2 in adult retinae is sufficient for retention of retinal axon regeneration was tested. Therefore, mice transgenic for the bcl-2 gene driven by the neuron-specific enolase promoter (Martinou, J-C. et al. Neuron 13, 1017-1030 (1994); Dubois-Dauphin, M., Frankowski, H., Tsujimoto, Y., Huarte, J. & Martinou, J-C. Proc. Natl. Acad. Sci. USA 91, 3309-3313 (1994)) were analyzed. The study was performed on line 73 of these transgenic mice. A series of timed matings was set up between males heterozygous for the transgene and wild-type (C57BL/6J) females. Half of the pups derived from these matings were transgenic. Cocultures of retinae and tecta derived from animals aged E14 through postnatal day 5 (P5, day of birth=P0), which covered the period before and after regenerative failure normally occurs were examined. As previously described, the experiment was designed so that retinal explants from each mouse had the possibility of being cocultured with tecta of wild-type or transgenic mice. Starting at E18, retinal explants from wild-type mice exhibited a failure of RGC axon elongation (n=15), regardless of whether confronted with wild-type or transgenic tectal tissues (FIG. 1C). The number of labeled retinal axons decreased 10-fold in comparison to E16 retinal explants. In contrast, when retinae were derived from bcl-2 transgenic animals, all retinal explants, harvested from animals aged E14 through P5, showed extensive fiber outgrowth (n=35) (FIG. 1C). No difference was observed in the numbers of retinal axons that invaded tectal slices derived from wild-type and bcl-2 transgenic mice. Therefore, constitutive expression of bcl-2 in RGCs, rather than in the CNS environment of the axon, overcomes regenerative failure of retinal axons in the perinatal period.

RGCs derived from bcl-2 transgenic mice retained the ability to grow axons throughout their life span. Extensive neurite outgrowth was observed from adult retinal explants of transgenic mice when they were cocultured with E16 tectal slices (n=10); the number of labeled retinal axons averaged 96.3±15.3, almost equivalent to the number obtained from an E16 retinotectal coculture. However, when the adult retinae were confronted with adult tectal tissues, little axonal growth was achieved (n=13) (FIG. 1B). This indicates that retinal axons of bcl-2 overexpressing mice have the ability to grow only into tissues expressing very permissive substrates, as presumably provided by the embryonic tectum. Therefore, bcl-2 is not the sole protein responsible for the regeneration of CNS axons in adult; it is probable that adult CNS contains inhibitory signals suppressing the regrowth of retinal axons from transgenic mice (Schnell, L. & Schwab, M. E. Nature 343, 269-272 (1990)). Thus, bcl-2 plays a central role in regulating the intrinsic genetic program for retinal axonal growth. Bcl-2 is essential but not sufficient for the regeneration of retinal axons in mature CNS under the conditions tested in this example (for this particular neural cell type and this particular bcl family member).

### EXAMPLE 4

### A bcl Family Member Promoted Axonal Growth in vivo

Subsequently, the regeneration of retinal axons in vivo was studied. Young pups (P4) obtained from the mating of males heterozygous for the bcl-2 transgene and C57BL/6J females, received a unilateral transection of the optic tract at the mid-tectal level. Axonal regrowth was assessed by tracing of retinal projection fibers with cholera toxin B-subunit (CT-B) (Angelucci, A., Clascá, F. & Sur, M. J. Neurosci. Meth. 65, 101-112 (1996)). To visualize the lesion site, every other sagittal section of these brains was collected for cresyl violet staining and reconstructed in three-dimensions with the Neurotrace program. In wild-type mice, the retinotectal projection was visible but was restricted to the tissue proximal to the lesion site (n=5). In contrast, axotomized retinal axons in transgenic mice grew in large numbers across the lesion site and innervated the tectum caudal to the injury (n=6). Thus, expression of bcl-2 in transgenic mice led to regeneration of retinal axons after optic tract transection in vivo. While in wild-type animals labeled axons did not cross the lesion site, those from bcl-2 transgenic mice regenerated across the lesion site and entered the caudal tectum. In three transgenic mice, the lesion produced a large, impassable gap in the superficial superior colliculus, but nevertheless the axons were observed to curve around the lesion site en route to the target tissue, without the addition of any bridging material or neurotrophic factors. Many axons reached the posterior border of the superior colliculus (SC). No axons were observed to invade the inferior colliculus. These results demonstrated that bcl-2 promoted retinal axon regeneration in vivo.

It should be emphasized that in the above examples, large numbers of RGCs in wild-type animals survived after injury, but seemed unable to regenerate their axons. Similar observations have been reported by other investigators (Misantone, L. J., Gershenbaum, M. & Murray, M. J. Neurocytol. 13, 449-465 (1984); Wikler, K. C., Kirm, J., Winderm, M. S. & Finlay, B. L. Dev. Brain Res. 28, 11-21 (1986); Harvey, A. R. & Robertson, D. J. Comp. Neurol. 325, 83-94 (1992))., who suggested a dissociation of neuronal survival and axonal regrowth after axotomy.

### EXAMPLE 5

### Effects of a bcl Family Member on Neuron Survival and Axonal Growth can be Distinguished in vitro

Whether these two activities of neurons, survival and axonal growth, can be separated in vitro was next examined. The anti-apoptotic function of bcl-2 is well established (Davies, A. M. TINS 18, 355-358 (1995); Korsmeyer, S. J. Immunol. Today 13, 285-288 (1992); Farlie, P. G., Dringen, R., Rees, S. M., Kannourakis, G. & Bernard, O. Proc. Natl. Acad. Sci. USA 92, 4397-4401 (1995); Bonfanti, L. et al. J. Neurosci. 16, 4186-4194 (1996)). Therefore, it is especially important to examine whether its grow-promoting activity is simply an indirect consequence of supporting cell survival. It has been suggested that bcl-2 suppresses apoptosis by impairing the activity of interleukin 1-converting enzyme (ICE) (Gagliardini, V. et al. Science 263, 826-828 (1994); Miura, M., Zhu, H., Rotello, R., Hartwieg, E. A. & Yuan, J. Cell 75, 653-660 (1993), a cysteine protease implicated as essential in the process of cell death in vertebrates Gagliardini, V. et al. Science 263, 826-828 (1994); Miura, M., Zhu, H., Rotello, R., Hartwieg, E. A. & Yuan, J. Cell 75, 653-660 (1993); Henkart, P. A. Immunity 4, 195-201 (1996); Nicholson, D. W. et al. Nature 376, 37-43 (1995). Use of a chemical that blocks ICE activity, presumably the same pathway that bcl-2 uses to suppress apoptosis, allowed testing of the relationship between the functions of axonal growth and cell survival. The capacity of an irreversible ICE-like protease inhibitor—ZVAD (Z-Val-Ala-Asp-CH₂F, Enzyme Systems Products) was investigated (Henkart, P. A. Immunity 4, 195-201 (1996); Nicholson, D. W. et al. Nature 376, 37-43 (1995); Fletcher, D. S. et al. J. Interferon Cytokin Res. 15, 243-248 (1995)—to influence the outgrowth of retinal axons. Using a dissociated cell culture system that allows visualization of single cell morphology, cultures were prepared from retinae of P2 pups. RGCs were prelabeled by injecting DiI into the tectum of P0 pups. Treatment with ZVAD at a concentration of 10 mM or above effectively reduced RGC death after 2 days in culture. Nevertheless. labeled RGCs from wild-type animals were round and devoid of neurites in culture (n=36), whereas, RGCs derived from bcl-2 transgenic mice (n=24) exhibited extensive axonal outgrowth. Note that this occurs in the absence of any neurotrophic factors added to the culture medium.

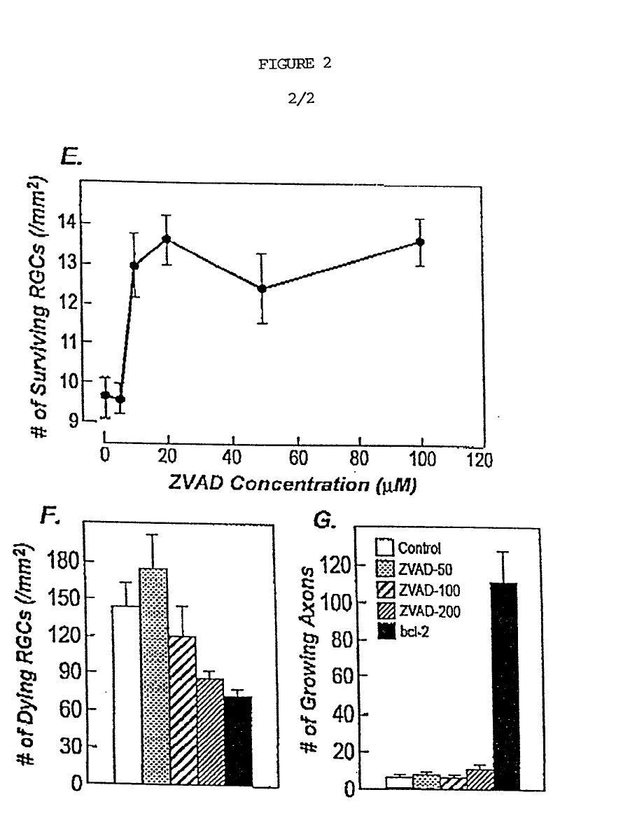

The effect of the ICE inhibitor was also tested in the explant coculture system with tissue prepared from wild-type P2 mice. Treatment with ZVAD reduced the extent of cell death in retinal explants (n=22) (FIG. 2A). A concentration of 200 (mM of ZVAD protected cells from death almost as well as bcl-2 in the transgenic mouse (n=6); however, the number of axons that invaded tectal slices was 10-fold less in cultures from wild-type animals than in those from bcl-2 transgenic mice (n=22) (FIG. 2B). While treatment with ZVAD was sufficient to prevent death of RGCs, it is not sufficient to promote axonal growth. By increasing the concentration of ZVAD, the number of dying cells in retinal explants decreased, whereas, the number of growing axons did not change significantly. Therefore, these examples suggested that cell survival and axonal growth are two distinct activities of RGCs; bcl-2, but not ICE inhibitors, supports both of these activities.

Evidence from other investigators (when viewed in conjunction with that provided herein) also support the theme that cell survival and axonal growth are two independent activities of neurons (Sagot, Y., Tan, S. A., Hammang, J. P., Aebischer, P. & Kato, A. C. J Neurosci. 16, 2335-2341 (1996); Dusart, I. & Sotelo, C. J Comp. Neurol. 347, 211-232 (1994)). The regenerative failure of retinal axons and the decrease of bcl-2 levels in RGCs occur (E18) before programmed cell death starts (P1-P5) (Young, R. W. J. Comp. Neurol. 229, 362-373 (1984)). The dissociation supports the observation from other investigators that the expression pattern of bcl-2 does not mirror recognized patterns of cell death in the CNS (Merry, D. E., Veis, D. J., Hickey, W. F. & Korsmeyer, S. J. Development 120, 301-311 (1994)); instead, it appears to correlate with cell differentiation and capacity for axonal outgrowth of neurons. Second, before programmed cell death begins, cell counts from spinal and facial motor neurons showed no significant difference in bcl-2 knockout mice and in wild-type animals (Michaelidis, T. M. et al. Neuron 17, 75-89 (1996)) whereas, a drastically reduced number of growing axons in cultures from bcl-2 knockout mice was found. Third, the ZVAD experiments further demonstrated that ICE inhibitor, though sufficient to block cell death, is not sufficient to support axonal growth. These all support the position that bcl-2 promotes axonal growth through a mechanism independent of its antiapoptotic activity.

Equivalents

Those skilled in the art will recognize, or be able to ascertain using no more than routine experimentation, numerous equivalents to the specific polypeptides, nucleic acids, methods, assays and reagents described herein. Such equivalents are considered to be within the scope of this invention and are covered by the following claims.

All of the above-cited references, issued patents and patent publications are hereby incorporated by reference. The contents of U.S. provisional application serial No. 60/021,713, filed on Jul. 12, 1996, are also specifically incorporated this reference.

What is claimed is:

1. A method of promoting axonal growth in a neural cell, comprising modulating the expression or bioactivity of a bcl family member in a neural cell such that axonal growth occurs.

2. The method of claim 1, wherein the cell is contacted with an agent which increases expression of a bcl family member.

3. The method of claim 1, wherein the cell is contacted with an agent which increases the bioactivity of a bcl family member.

4. The method of claim 1, wherein the bcl family member is bcl-2.

5. The method of claim 1, wherein the step of modulating occurs in vivo.

6. The method of claim 5, further comprising testing agents which influence the ability of a bcl-2 modulating agent to promote axonal growth.

7. The method of claim 1, wherein the neural cell is in the central nervous system.

8. The method of claim 7, wherein the neural cell is in the ascending tract of the spinal cord.

9. The method of claim 7, wherein the neural cell is in the brain.

10. The method of claim 7, wherein the neural cell is in the peripheral nervous system.

11. The method of claim 1, wherein the bcl-2 family member is a bcl polypeptide or fragment thereof.

12. The method of claim 1, wherein the bcl family member is a polypeptide comprising the BH1 and BH2 domains of a bcl-2 polypeptide.

13. The method of claim 1, further comprising additionally administering an agent which creates an environment favorable to axonal cell growth.

14. The method of claim 13, wherein the agent comprises one or more agents selected from the group consisting of: trophic factors, receptors, extracellular matrix proteins, intrinsic factors, or adhesion molecules.

15. A method of treating a subject that has suffered a traumatic injury in which nerve cell injury has occurred, comprising administering to said subject a bcl modulating agent such that treatment of the traumatic injury occurs.

16. A method of treating a subject for a state characterized by diminished potential for axonal growth, comprising administering a therapeutically effective amount of an agent which modulates the bioactivity or expression of a bcl family member in a subject such that axonal growth occurs.

17. The method of claim 16, wherein the agent increases expression of a bcl family member.

18. The method of claim 16, wherein the agent increases the bioactivity of a bcl family member.

19. The method of claim 16, wherein the state characterized by diminished potential for axonal growth is a central nervous system disorder.

20. The method of claim 19, wherein the state characterized by diminished potential for axonal growth is a traumatic injury to the central nervous system.

21. The method of claim 16, wherein the state characterized by diminished potential for axonal growth is a peripheral nervous system disorder.

22. The method of claim 16, wherein the bcl family member is a bcl-2 polypeptide or fragment thereof.

23. The method of claim 16, wherein the bcl family member is a polypeptide comprising the BH1 and BH2 domains of a bcl polypeptide.

24. The method of claim 16, further comprising additionally administering an agent which creates an environment favorable to axonal cell growth.

25. The method of claim 24, wherein the agent comprises one or more agents selected from the group consisting of: trophic factors, receptors, extracellular matrix proteins, or intrinsic factors.

26. A method of treating a state characterized by diminished potential for axonal growth, comprising administering to a subject with said state a therapeutically effective amount of a gene construct for expressing a bcl-2 family member, wherein the gene construct is formulated for delivery into neural cells of the subject such that axonal growth occurs.

27. The method of claim 26, wherein the subject is a mammal.

28. The method of claim 26, wherein the subject is a human.

29. The method of claim 26, wherein the gene construct is in a viral vector.

30. The method of claim 29, wherein the viral vector is an adenovirus.

31. The method of claim 29, wherein the viral vector is a herpes virus.

32. The method of claim 26, wherein the gene construct is formulated in liposomes.

33. The method of claim 26, wherein the gene construct is in a gene delivery composition specially formulated to cross the blood-brain barrier.

34. The method of claim 26, wherein the neural cell of the subject is in the central nervous system.

35. The method of claim 34, wherein the neural cell is in the spinal cord.

36. The method of claim 34, wherein the neural cell is in the brain.

37. The method of claim 26, wherein the neural cell is in the peripheral nervous system.

38. The method of claim 26, wherein the bcl family member is a bcl-2 polypeptide or fragment thereof.

39. The method of claim 26, wherein the bcl family member is a polypeptide comprising the BH1 and BH2 domains.

40. The method of claim 26, further comprising further administering an agent which creates an environment favorable to axonal cell growth.

41. The method of claim 40, wherein the agent comprises one or more agents selected from the group consisting of: trophic factors, receptors, extracellular matrix proteins, or intrinsic factors.

42. A pharmaceutical preparation comprising a therapeutically effective amount of a recombinant transfection system for treating a state associated with diminished potential for axonal growth in a subject, comprising
(i) a gene construct including the nucleic acid encoding a bcl family member;
(ii) a gene delivery composition for delivering said gene construct to a neural cell of the subject and causing the cell to be transfected with said gene construct resulting in expression thereof; and further comprising
(iii) one or more agents favorable for the promotion of axonal growth.

43. The pharmaceutical preparation of claim 42, wherein the agent is selected from the group consisting of: trophic factors, receptors, extracellular matrix proteins, intrinsic factors, or adhesion molecules.

44. The preparation of claim 42, wherein the gene delivery composition is selected from the group consisting of a recombinant viral particle, and a plasmid.

45. The preparation of claim 42, wherein the gene delivery composition has been specially formulated to cross the blood-brain barrier.

46. A packaged drug for treating a state associated with diminished potential for axonal growth, comprising a bcl-2 modulating agent packaged with instructions for treating a subject having said state.

47. The packaged drug of claim 46, wherein the bcl modulating agent increases expression of a bcl family member.

48. The packaged drug of claim 47, wherein said drug is used to increase expression of a bcl family member in a neural cell of the central nervous system.

49. The packaged drug of claim 48, wherein said drug is used to increase expression of a bcl family member in a neural cell of the spinal cord.

50. The packaged drug of claim 48, wherein said drug is used to increase expression of a bcl family member in a neural cell of the brain.

51. The packaged drug of claim 47, wherein said drug is used to increase expression of a bcl family member in the peripheral nervous system.

52. The packaged drug of claim 47, wherein the bcl family member is a bcl-2 polypeptide or fragment thereof.

53. The packaged drug of claim 47, wherein the bcl family member is a polypeptide comprising the BH1 and BH2 domains of a bcl-2 polypeptide.

54. The packaged drug of claim 47, further comprising an agent which creates an environment favorable to axonal cell growth.

55. The packaged drug of claim 54, wherein the agent comprises one or more agents selected from the group consisting of: trophic factors, receptors, extracellular matrix proteins, intrinsic factors, or adhesion molecules.

56. The packaged drug of claim 47, wherein the bcl modulating agent is a pharmaceutical preparation comprising a bcl-2 gene in a plasmid.

57. The packaged drug of claim 47, wherein the bcl modulating agent is a pharmaceutical preparation comprising a bcl-2 gene in a viral vector.

58. The packaged drug of claim 47, wherein the bcl modulating agent is a pharmaceutical preparation comprising a bcl-2 gene in a non-viral delivery system.

59. A method for selecting an agent for its ability to promote axonal growth in a culture comprising;
(i) contacting a first tissue sample comprising axons with a second tissue sample into which said axons can grow;
(ii) modulating the expression of a bcl family member in the first tissue sample; and
(iii) determining whether axonal growth occurs.

60. A method for selecting an agent for its ability to promote axonal growth in a culture comprising;
(i) forming a culture by contacting a first tissue sample comprising axons with a second tissue sample into which said axons can grow;
(ii) contacting said culture with a test agent, and
(iii) determining whether axonal growth occurs.
